(19) United States
(12) Patent Application Publication
(10) Pub. No.: US 2017/0157010 A1
(43) Pub. Date: Jun. 8, 2017

(54) METHODS AND COMPOSITIONS FOR TREATMENT OF DISORDERS ASSOCIATED WITH DAMAGE INDUCED BY FREE RADICALS

(71) Applicants: Margo P. Cohen, New York, NY (US); Clyde W. Shearman, West Chester, PA (US)

(72) Inventors: Margo P. Cohen, New York, NY (US); Clyde W. Shearman, West Chester, PA (US)

(73) Assignee: Glycadia, Inc., Philadelphia, PA (US)

(21) Appl. No.: 14/962,030

(22) Filed: Dec. 8, 2015

(51) Int. Cl. A61K 8/41; A61K 9/00; A61Q 19/00; A61K 31/196

(52) U.S. Cl. CPC A61K 8/411; A61K 31/196; A61K 9/0019; A61K 9/0053; A61K 9/0014; A61Q 19/004; A61K 2800/74

(57) ABSTRACT

The present invention relates to treatment of various disorders, diseases or conditions associated with or mediated by oxidative stress arising from an imbalance between the production of and the ability to neutralize reactive free radicals. The present invention is directed to methods and compositions capable of scavenging reactive free radicals for therapeutically or prophylactically treating disorders associated with free radical-induced damage, said compositions comprising an amount of 2-(3-chlorophenylamino) phenylacetic acid (23CPPA) or a pharmaceutically acceptable salt thereof that interacts with free radicals generated by cellular metabolism or ultraviolet radiation. In some embodiments, the composition comprises pharmaceutically or cosmetically acceptable adjuncts. The present invention further provides one or more kits that are useful for delaying, treating or preventing the consequences of aging and free radical damage on the condition or appearance of the skin.

### FIELD OF INVENTION

The present invention is directed to methods and compositions using 2-[(3-chlorophenyl) amino] phenylacetic acid (23CPPA) in the prevention and treatment of various disorders associated with oxidative stress arising from an imbalance between the production and neutralization of reactive free radicals.

### BACKGROUND OF THE INVENTION

The present invention relates to treatment of various disorders, diseases or conditions associated with or mediated by oxidative stress arising from an imbalance between the production of and the ability to neutralize reactive free radicals. The present invention is directed to methods and compositions capable of scavenging reactive free radicals for therapeutically or prophylactically treating disorders associated with free radical-induced damage. More particularly it has been discovered that 2-[(3-chlorophenyl) amino] phenylacetic acid (23CPPA) interacts with free radicals generated by cellular metabolism or ultraviolet radiation, and that compositions compromising an amount of or a pharmaceutically acceptable salt thereof are useful in the treatment of disorders associated with free radical damage. In some embodiments, the composition comprises pharmaceutically or cosmetically acceptable adjuncts. The present invention further provides one or more kits that are useful for delaying, treating or preventing the consequences of aging and free radical damage on the condition or appearance of the skin.

23CPPA has been shown to impede the condensation of free glucose with albumin, and thereby decrease the formation of albumin modified by Amadori glucose adducts, lowering the concentration and lessening the pathophysiological effects of Amadori-modified albumin (U.S. Pat. No. 6,355,680). 23CPPA acid does not have the molecular formula of a nonsteroidal anti-inflammatory drug (NSAID), is not an isomer or enantiomer of a NSAID, and is not a pharmacologic inhibitor of the cyclooxygenase (COX) enzymes COX-1 and COX-2. 23CPPA has no substituent in the ortho position and is structurally different from the nonsteroidal anti-inflammatory diclofenac, an agent that can absorb ultraviolet light (U.S. Pat. No. 3,652,762). The free radical scavenging and ultraviolet radiation absorption properties of 23CPPA are of independent use in conditions in which Amadori-modified albumin does not play a causative role, such as skin disorders of aging and ultraviolet radiation, scleroderma, rheumatoid arthritis and Parkinson's disease. Increased levels of this analyte are not found in the skin of patients with scleroderma (Hettema et al, Int J Rheumatol 2011, doi 10.1155/2011/417813), or in skin or other relevant tissues in the various disorders noted above in which oxidative stress with free radical generation plays a causative role. Ultraviolet radiation causes damage to DNA by generation of highly reactive chemical intermediates, such as hydroxyl and oxygen radicals, and by UV photon induced formation of pyrimidine dimers (Anwar, Clin in Derm 2004; 22; 189-196). Ultraviolet radiation also can damage collagen fibers and thereby accelerate aging of the skin (Fisher et al, New Engl J Med 337:1419-1428, 1997). Formation of protein aggregates in Lewy bodies of patients with Parkinson's disease is consequent to and a marker of oxidative stress, and the ability of antioxidants to block oxidative stress-dependent neurodegeneration indicates that release of free radicals by activated microglia antecedes Lewy body protein aggregation (Koutsilieri et al, Journal Neurology 249Supp12:ii1-5, 2002.) Oxidative stress precedes and is a cause of protein aggregation that gives rise to formation of Lewy bodies which may show immunoreactivity to reactant products generated as a consequence of oxidative stress.

Oxygen is the most electronegative element with the highest reduction potential in biological systems. Metabolic pathways in mammalian cells utilize oxygen as the ultimate oxidizing agent to harvest free energy but also generate various oxygen centered free radicals. Free radicals are reactive chemical species of atoms or groups of atoms that have unpaired electrons in their outer orbitals and that are formed when oxygen interacts with certain molecules. The unpaired electron renders the free radical highly reactive because it captures electrons from other molecules. Free radicals are oxidizing agents that cause oxidation of other molecules as they donate their electrons to free radicals. Once formed these highly reactive radicals can damage cellular components and initiate inflammatory and other pathways that contribute to various disease entities including damage to the skin and from aging, auto-immune, neurodegenerative, and inflammatory disorders.

The main oxygen-centered radicals associated with cell damage are superoxide, hydroxyl radical and nitric oxide. Other molecules such as hydrogen peroxide and peroxynitrate, while not in themselves free radicals, can respectively generate free radicals through metal-facilitated conversion and hydrogenated breakdown. Free radicals and related molecules such as hydrogen peroxide and peroxynitrate are together referred to as reactive oxygen species, signifying their ability to promote oxidative changes. Although enzymes such as superoxide dismutase, catalase, glutathione peroxidase and glutathione reductase help counter free radical-induced damage, as do antioxidants such as vitamin A, vitamin C and vitamin E, there remains a need for compounds capable of scavenging free radicals, aborting activation of the direct or indirect pathways leading to cellular damage and delaying progression of the disorder. Reduction of free radicals without or with combinatorial treatments is a suitable strategy to therapeutically or prophylactically treat these disorders.

Oxidative stress is an imbalance between the production of reactive oxygen species and physiological antioxidant defenses to neutralize these radicals. It causes cellular damage because reactive oxygen species oxidize critical cell components such as proteins, lipids and DNA. Oxidative stress occurs frequently in conditions characterized by immune activation and inflammation and is associated with diverse disorders including damage to the skin and from aging, auto-immune disease such as scleroderma and rheumatoid arthritis, and Parkinson's disease.

Strategies for preventing free-radical induced damage and oxidative stress include inhibiting the formation of free radicals and promoting the catalytic removal or the scavenging of oxyradicals once they are formed. For example, chelators such as desferrioxamine inhibit iron-catalyzed superoxide generation and oxyradical formation, superoxide dismutase and catalase promote catalytic removal of superoxide, and various small molecules including congeners of glutathione are capable of noncatalytically scavenging hydroxyl radicals (van der Kraaij et al, Circulation, 80:158, 1989; Gutteridge et al, Biochem J, 184:469, 1979; Mitsos et al, Circulation 73:1077, 1986). However, existing compounds and methods have drawbacks such as poor penetration of brain and the need for parenteral administration or high dosages, and there is a need for small molecules that are efficient scavengers of oxyradicals that can be administered orally and can penetrate the blood brain barrier. 23CPPA is a small molecule with properties that address this need.

The present invention relates to the discovery that 23CPPA possesses intrinsic properties of free radical scavenging and the absorption of ultraviolet radiation and is therefore of use in the prevention and treatment of disorders causally related to reactive free radicals, such disorders including damage to the skin due to aging and from solar radiation, auto-immune disease such as scleroderma and rheumatoid arthritis, and neurodegenerative disease such as Parkinson's disease.

The aging process causes changes in the skin characterized in part by wrinkles, altered pigmentation, loss of skin tone, and alterations in the collagenous extracellular matrix of connective tissue. Exposure to ultraviolet radiation from sunlight accelerates this process and causes premature aging of the skin which has similar characteristics. The sun emits ultraviolet radiation in the UVA (315-400 nm), UVB (280-315 nm), and UVC (190-280 nm) bands, with UVA radiation being the predominant form reaching the earth's surface. Solar UVB and UVA radiations have a capacity to generate reactive chemical species, including free radicals, in cells. UVA, UVB, and UVC can all damage collagen fibers and thereby accelerate aging of the skin. Histological manifestations include disorganization and fragmentation of collagen fibrils, changes in cross-links, and abnormal accumulation of elastin-containing material (Fisher et al, New Engl J Med, 337:1419, 1997; Yamauchi et al, J Invest Dermatol, 97:938, 1991). Both UVA and UVB destroy vitamin A in skin, which may cause further damage. UVA can contribute to skin cancer via DNA damage through production of free radicals and reactive oxygen species. UVB and UVC generate highly reactive chemical intermediates, such as hydroxyl and oxygen radicals, which in turn also can damage DNA.

Scleroderma is a disorder of unknown etiology which is characterized by increased vasoreactivity and vascular alterations, tissue fibrosis in the skin and/or internal organs, and autoantibodies against various cellular antigens. Reactive oxygen species promote the auto-immune response by inducing cleavage and fragmentation of auto-antigens, giving rise to revelation of immunocryptic epitopes in self antigens that can initiate auto-immunity (Sambo et al, Arthritis & Rheumatism, 44:2653, 2001). Several of the auto-antigens in scleroderma have metal binding sites and are uniquely susceptible to cleavage by reactive oxygen species and are capable of focusing metal-catalyzed oxidation reactions and consequent fragmentation to reveal cryptic auto-antigenic structures (Casciola-Rosen et al, J Exp Med 185:71, 1997). Enhanced production of reactive oxygen radicals and oxidative stress play an important role in the pathogenesis of scleroderma as evidenced for example by increased plasma levels of free radical activity, increased susceptibility of lipoproteins to oxidation, and low serum levels of anti-oxidants such as ascorbic acid. Monocytes from patients with scleroderma generate increased amounts of superoxide anion compared to normal controls, promoting oxidative stress and downstream activation of inflammatory cytokines that alter vascular function (Sambo et al, J Invest Dermatol 112:78, 1999).

Rheumatoid arthritis is a chronic inflammatory joint disease characterized by painful swelling and typically affecting the small joints of the hands and feet. It is an auto-immune disorder in which the immune system mistakenly attacks self-antigens. Free radicals can directly and indirectly damage articular constituents leading to the clinical expression of inflammatory arthritis, such damage including degradation and inhibition of production of proteoglycans, activation of collagenases and fragmentation of collagen, acceleration of bone resorption, cartilage and bone destruction, induction of proinflammatory cytokines, and consumption of ascorbate in the synovial fluid, a lowered level of anti-oxidants, and accentuation of oxidant/anti-oxidant imbalance in chondrocytes of patients with rheumatoid arthritis (Heliovaara et al, Ann Rheumatic Dis 53:51, 1994; Bates et al, Ann Rheumatic Dis 43:462, 1984; Mazzetti et al, Clinical Sci 101:593, 2001).

Parkinson's disease is a movement disorder characterized symptomatically by resting tremor, bradykinesia, rigidity and postural instability, and pathologically by progressive degeneration of neurons located in the substantia nigra, a part of the midbrain. These neurons synthesize and secrete the neurotransmitter dopamine, and the loss of dopaminergic influence on structures in the basal ganglia leads to the classic symptoms of Parkinson's disease. Free radicals are important agents responsible for the destruction of substantia nigra neurons that leads to Parkinson's disease. Substantia nigra neurons are susceptible to free radical-induced damage that can cause degeneration and death. Dopamine can be oxidized by the enzyme monoamine oxidase B or can undergo auto-oxidation to give rise to hydrogen peroxide that, in the absence of adequate amounts of glutathione or presence of excess iron, both of which are found in patients with Parkinson's disease, produces excess free radicals (Ciccone, Physical Therapy 78:313, 1998; Sofic et al, Neurosci Lett 142:128, 1992; Spina & Cohen, Proc Natl Acad Sci 86:1398, 1989; Sudha et al, Neurol India 51:60, 2003).

This invention should not be construed as being limited solely to these examples of disorders associated with free-radical damage, and one of skill in the art would appreciate that 23CPPA may be useful, alone or in combination, to therapeutically or prophylactically treat disorders and diseases arising from oxidative stress and free radical damage.

The present invention therefore addresses the existing need for compounds and compositions capable of scavenging reactive free radicals for therapeutically or prophylactically treating disorders associated with free radical-induced damage and that can deliver the active agent across the blood-brain barrier.

### SUMMARY OF THE INVENTION

It is an object of the present invention to provide novel methods and compositions for the alleviation of oxidative stress associated with an imbalance between the production of reactive oxygen species and physiological antioxidant defenses to neutralize these radicals.

It is another object of the present invention to provide novel methods and compositions for therapeutically or prophylactically treating disorders associated with free radical-induced damage. The method includes the step of administering to a patient in need of such treatment a composition comprising the compound 23CPPA or a pharmaceutically acceptable salt thereof in an amount sufficient to elicit a therapeutic or prophylactic effect.

In some embodiments, these and other aspects of the invention are achieved with the discovery that the free radical scavenging and ultraviolet absorption properties of 23CCPA acid do not require anti-glycation activity and that the compound is therefore of use in diseases associated with free radical damage, without regard to glycation-associated pathology.

In some embodiments, these and other aspects of the invention are achieved with the discovery that free radical scavenging and ultraviolet absorption properties of 23CPPA therapeutically and prophylactically address disorders in which damage induced by free radicals and ultraviolet radiation, are causally contributory, such disorders including skin damage due to aging and solar radiation, auto-immune disease such as scleroderma and rheumatoid arthritis and neurodegenerative disease such as Parkinson's disease.

In some embodiments, these and other aspects of the invention are achieved with one or more kits that for delaying, treating or preventing the consequences of aging, ultraviolet exposure, and free radical damage on the condition or appearance of the skin.

### DETAILED DESCRIPTION OF THE INVENTION

It has been unexpectedly discovered as described in the present invention that the compound 23CPPA and its pharmaceutically acceptable salts possess the intrinsic ability to scavenge reactive free radicals and thereby modulate the imbalance between the production of reactive oxygen species and physiological antioxidant defenses to neutralize these radicals.

It has been unexpectedly discovered as described in the present invention that the compound 23CPPA and its pharmaceutically acceptable salts possess the intrinsic ability to absorb ultraviolet radiation and thereby modulate damage to the skin arising from exposure to ultraviolet radiation from the sun, which generates highly reactive chemical intermediates, such as hydroxyl and oxygen radicals, which injure components of the skin and can damage DNA.

It is a novel and unanticipated finding of the present invention that the free radical scavenging activity of the active agent 23CPPA is an inherent property of this compound.

It is a novel and unanticipated finding of the present invention that the ultraviolet radiation absorbing properties of the active agent 23CPPA is an inherent property of this compound.

It is an object of this invention to provide a method for treating disorders in which free radical damage is causally contributory, such disorders including skin damage due to aging and solar radiation, auto-immune disease such as scleroderma and rheumatoid arthritis and neurodegenerative disease such as Parkinson's disease.

It is another object of this invention to provide a method for treating disorders of the skin associated with damage from solar radiation.

It is another object of this invention to provide a method for treating disorders of the skin associated with damage to the skin from aging.

This invention also provides therapeutic compositions comprising the above described compound.

This invention further provides a method for treating disorders associated with free radical damage comprising administering to a patients with such a disorder an effective amount of a therapeutic composition comprised of 23CPPA capable of scavenging reactive free radicals and a pharmaceutically acceptable carrier.

It is also an object of this invention to provide a method for treating disorders associated with solar radiation comprising an effective amount of a therapeutic composition comprised of 23CPPA capable of absorbing ultraviolet radiation and a pharmaceutically acceptable carrier.

The present invention further provides one or more kits that are useful for delaying, treating or preventing the consequences of aging and free radical damage on the condition or appearance of the skin.

The present invention comprises one or more compositions containing 23CPPA or a pharmaceutically acceptable salt thereof formulated into compositions together with one or more non-toxic physiologically acceptable carriers, adjuvants or vehicles which are collectively referred to herein as carriers, for parenteral injection, for oral administration in solid or liquid form, and for topical administration.

Compositions suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol and the like), suitable mixtures thereof, vegetable oils (such as olive oil) and injectable organic esters such as ethyl oleate. Proper fluidity can be maintained, for examples, by use of coating such as lecithin, by maintenance of the required particle size in the case of dispersions and by use of surfactants. These compositions may also contain adjuvants such as preserving, wetting, emulsifying and dispensing agents.

Solid dosage forms for oral administration include capsules, pills, tablets, powders and granules. In such solid dosage forms, the active compound is admixed with at least one inert customary, pharmaceutically acceptable excipient or carrier such as sodium citrate or dicalcium phosphate or (a) fillers or extenders, as for example starches, lactose, sucrose, glucose, mannitol and silicic acid; (b) binders, as for example carboxymethylcellulose, alginates, gelatin, polyvinylpyrrolidone, sucrose and acacia; (c) humectants, as for example agar-agar, calcium carbonate, potato or tapioca starch, alginic acid, certain complex silicates and sodium carbonate; (d) solution retarders, as for example paraffin, absorption accelerators, as for example quaternary ammonium compounds; (e) wetting agents, as for example cetyl alcohol and glycerol monostearate; (f) adsorbents, as for examples kaolin and bentonite; and (g) lubricants, as for example talc, calcium stearate, magnesium stearate, solid polyethylene glycols, sodium lauryl sulfate or mixtures thereof. In the case of capsule, tablets and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols and the like. Solid dosage forms such as tablets, capsules, pills and granules can be prepared with coatings and shells, such as enteric coating and others known in the art. They may contain opacifying agents and can also be of such composition that they release the active compound or compounds in a certain part of the intestinal tract in a delayed manner. Examples of embedding compositions which can be used are polymeric substances and waxes.

Dosage forms for topical administration of the method of this invention include lotions, ointments, creams, gels, liniments, pastes, solutions, powders suspensions and sprays. The active component is admixed under sterile conditions with a physiologically acceptable carrier and any preservatives, buffers or propellants as may be required. Topical compositions may also contain moisturizers, humectants, demulcents, oils, water, emulsifiers, thickeners, surface active agents, fragrances, preservatives, hydrotropic agents, chelating agents, vitamins, minerals, permeation enhancers, cosmetic adjuvants, depigmentation agents, foaming agents, conditioners, viscosifiers, buffering agents and sunscreens.

Actual dosage levels of active ingredients in the compositions of the present invention may be varied so as to obtain an amount of the active ingredient that is effective to obtain a desired therapeutic response for a particular composition and method of administration. The selected dosage level depends on the desired therapeutic effect, route of administration, duration of treatment and other factors. The total daily dose of the method and compositions of this invention administered to a host in single or divided dose may be in amounts of 1 mg/kg to 20 mg/kg of body weight when administered parenteral or orally and may be from lower to higher values when administered topically. Dosage unit compositions may contain such amounts or such submultiples therefor as may be used to make the daily dose. It will be understood, however, that the specified dose level for any particular patient will depend on a variety of factors including the body weight, general health, gender, diet, time and route of administration, rates of absorption and excretion, combination with other drugs and the severity of the particular disease being treated. The dosage level may also depend on patient response as determined by symptoms and signs of the disease for which the treatment is administered.

The above disclosure generally describes the present invention. A more complete understanding can be obtained by reference to the following specific examples, which are provided herein for purposes of illustration only and are not intended to limit the scope of the invention.

### Example 1

### 223CPPA Scavenges Free Radicals

A radical cation of 2,2′-azino-bis(3-ethylbenzthiazoline-6-sulfonic acid) diammonium salt (ABTS) was generated by oxidation with manganese dioxide, forming the blue-green radical cation ABTSY+ with spectrophotometric absorbance at 734 nanommeters (nm). The reaction was initiated by the addition of 23CPPA (5-40 micromolar) to ABTSY+ (450 micromolar) in phosphate buffer (75 millimolar, pH 7.4) and decay of the blue-green radical cation absorbance was monitored over time. Absorbance profiles were obtained with incubation in phosphate buffer (75 millimolar, pH 7.4) as control. The reaction of anti-oxidants with stable free radicals such as ABTSY+ that is generated from ABTS measures their free radical scavenging capacities. The Table depicts change with time of the absorbance at 734 nm of ABTSY+ after addition of 23CPPA to buffer compared to buffer alone, demonstrating progressive bleaching of ABTSY+ by 23CPPA and, therefore, free radical scavenging activity of 23CPPA.

| | Decay (% of starting absorbance) | | | | |
|---|---|---|---|---|---|
| | 23CPPA (micromolar) | | | | |
| Minutes | 0 | 5 | 10 | 20 | 40 |
| 1 | 99 | 74 | 57 | 37 | 20 |
| 2 | 97 | 72 | 56 | 27 | 20 |
| 3 | 95 | 69 | 53 | 23 | 3 |
| 4 | 94 | 67 | 52 | 20 | 1 |
| 5 | 94 | 65 | 51 | 17 | 0 |
| 6 | 93 | 63 | 50 | 14 | 0 |
| 7 | 93 | 61 | 48 | 13 | 0 |
| 8 | 92 | 60 | 47 | 12 | 0 |
| 9 | 92 | 58 | 46 | 12 | 0 |
| 10 | 92 | 56 | 44 | 11 | 0 |
| 11 | 91 | 55 | 44 | 10 | 0 |
| 12 | 91 | 53 | 43 | 10 | 0 |
| 13 | 90 | 52 | 43 | 9 | 0 |
| 14 | 90 | 51 | 42 | 9 | 0 |
| 15 | 89 | 49 | 42 | 9 | 0 |

### Example 2

### 23CPPA Absorbs Ultraviolet Radiation

Ultraviolet (UV) absorption peaks of 2-(3-chlorophenylamino)phenylacetic acid at 211 and 283 nm, encompassing wavelength range of medium wave UVB (315-280 nm) and short wave UVC (280-190 nm).

| Wavelength (nm) | Absorbance |
|---|---|
| 190 | 0.5 |
| 195 | 0.75 |
| 197 | 1.0 |
| 198 | 1.25 |
| 199 | 1.5 |
| 205 | 2.0 |
| 208 | 2.1 |
| 211 | 2.2 |
| 212 | 2.0 |
| 220 | 1.25 |
| 222 | 1.0 |
| 232 | 0.5 |
| 235 | 0.45 |
| 239 | 0.43 |
| 243 | 0.43 |
| 249 | 0.43 |
| 252 | 0.43 |
| 256 | 0.43 |
| 260 | 0.5 |
| 272 | 0.75 |
| 283 | 0.93 |
| 291 | 0.75 |
| 300 | 0.5 |
| 308 | 0.25 |
| 325 | 0 |

### Example 3

### Bioavailability of 23CPPA

Male rats were given a single dose of 23CPPA by the oral route (30 mg/kg) or by the intravenous route (3.0 mg/kg). Timed samples of blood were collected before and after dosing, and plasma concentrations of the compound were determined with liquid chromatography mass spectrometry (LC-MS-MS) analysis. Oral bioavailability, calculated from the plasma concentrations after oral versus intravenous administration, was 85%, indicating that the drug is absorbed from the gastrointestinal tract and enters the circulation for systemic delivery.

### Example 4

### 23CPPA Penetrates Brain Following Oral Administration in Rats

Rat hemi brains were obtained 1,4 and 6 hours after administration by gavage of the sodium salt of 23CPPA at doses of 15 and 60 mg/kg and were mixed with two equivalents of water and homogenized. The resulting homogenate was extracted with acetonitrile containing internal standard and concentrations of the compound were analyzed by high pressure liquid chromatography/two stage mass spectrometry (HPLC/MS/MS). Plasma samples also were collected from the same rats at these time points for measurement of plasma concentrations of the compound. Brain concentrations were dose proportional and showed an approximately 3:1 molar ratio with plasma concentrations, indicating excellent penetration of the compound into the brain.

| Dose | Time | Brain | Blood |
|---|---|---|---|
| (mg/kg) | after Dose | (micromolar) | (micromolar) |
| 15 | 1 hour | 76 | 27 |
| 15 | 4 hours | 12.4 | 4 |
| 15 | 6 hours | 11.9 | 3 |
| 60 | 1 hour | 148 | 57 |
| 60 | 4 hours | 61 | 28 |
| 60 | 6 hours | 45 | 22 |

### Example 5

### 23CPPA Reduces UV-Induced Collagen Cross Linking

Oxidative change of type I collagen was induced by exposure to ultraviolet (UV) radiation (325 nm) conducted for 24 hours at 25° C. in the absence or presence of 23CPPA (10:1 molar ratio of 23CPPA to type I collagen). Samples were frozen and lyophilized, the residue was dissolved in 0.1 ml of 70% formic acid, cyanogen bromide (CnBr) (20:1 volume:volume) was added, and the solutions were incubated at 30° C. for 18 hours. Samples were spin filtered into 0.125M Tris, pH 6.8, containing 2% sodium dodecyl sulfate (SDS) and 2% glycerol using a filter with a molecular weight cutoff of 10,000. The extent of collagen cross linking was determined by analyzing equal volumes of samples subjected to SDS-polyacrylamide gel electrophoreses (SDS-PAGE) (15% Tris-HCl gel) and assessing migration of the CnBr peptides. CnBr peptides less than 10,000 molecular weight, prepared from UV-treated collagen in either the absence or presence of 23CPPA, escaped from the samples during the spin filter due to the molecular weight cut-off limits of the filter, with retention of CnBr peptides >10,000 molecular weight. SDS-PAGE analysis showed that CnBr peptides of higher molecular weight were produced when 23CPPA was not present during exposure to UV radiation. CnBr peptides of type I collagen treated with UV radiation in the presence of 23CPPA acid were of lower molecular weight than CnBr peptides of collagen treated with UV radiation in the absence of 23CPPA.

### SDS PAGE Electrophoresis

| | | CnBr | | Molecular |
|---|---|---|---|---|
| Sample | UV | Digestion | 23CPPA | Weight |
| Type I collagen | Yes | No | No | 180,000 |
| Type I Collagen | Yes | Yes | No | 85,000 |
| Type I Collagen | Yes | Yes | Yes | 65,000 |

### Example 6

### 23CPPA Decreases UV-Induced Changes in Type I Collagen Fluorescence Emission.

Type I collagen was solubilized in 1.5% acetic acid in the absence or presence of 23CPPA (10:1 molar ratio) and was subjected to UV radiation at for 3 to 24 hours at 25° C. The intensity of the fluorescence emission peak at excitation 380 nm, which is due to pepsin-digestible cross-links, decreases with UV radiation; this decrease was lessened in the presence of 23CPPA during exposure to the UV radiation.

| UV Exposure | Relative fluorescence | |
|---|---|---|
| (hours) | 23CPPA Absent | 23CPPA Present |
| 0 | 100% | 100% |
| 3 | 95% | 120% |
| 6 | 75% | 120% |
| 24 | 60% | 85% |

What is claimed is:

1. A method of treating disorders associated with free radical damage, said method comprising administering an effective amount of the compound 2-(3-chlorophenylamino) phenylacetic acid or a pharmaceutically or cosmetically acceptable salt thereof.

2. The method of claim 1 wherein said disease or disorder is selected from the group consisting of skin aging, skin wrinkling, hyperkeratosis, and skin solar damage.

3. The method of claim 1 wherein said disease or disorder is selected from the group consisting of scleroderma, rheumatoid arthritis, and auto-immune disease.

4. The method of claim 1 wherein said disease or disorder is selected from the group consisting of Parkinson's disease and neurodegenerative disease.

5. The method of claim 1 wherein said compound is administered from the group consisting of topical, oral, intramuscular, intravenous and subcutaneous dosage forms.

6. The method of claim 5 wherein said topical dosage form is selected from the group consisting of a lotion, a cream, a gel, a liniment, an ointment, a paste, a solution, a powder and a suspension.

7. The method of claim 5 wherein said oral dosage form is selected from the group consisting of a tablet, a capsule, a pill, powder and granules.

8. The method of claim 5 wherein said intramuscular, intravenous and subcutaneous dosage forms are selected from the group consisting of physiologically acceptable sterile aqueous or nonaqueous solutions, suspensions or emulsions and sterile powders for reconstitution into sterile injectable solutions or dispersions.

9. The method of claim 6 wherein said topical dosage form contains a pharmaceutically acceptable carrier.

10. The method of claim 7 wherein said oral dosage form contains a pharmaceutically acceptable carrier.

11. The method of claim 8 wherein said intramuscular, intravenous and subcutaneous dosage forms contain a pharmaceutically acceptable carrier.

12. The method of claim 9 wherein said pharmaceutically acceptable carrier further comprises selections from the group consisting of a moisturizer, a humectant, a demulcent, oil, water, an emulsifier, a thickener, a surface active agent, a fragrance, a preservative, an antioxidant, a hydrotropic agent, a chelating agent, a vitamin, a mineral, a permeation enhancer, a cosmetic adjuvant, a depigmentation agent, a foaming agent, a conditioner, a viscosifier a buffering agent and a sunscreen.

13. The method of claim 10 wherein said pharmaceutically acceptable carrier further comprises selections from the group consisting of inert excipients, fillers, extenders, binders, humectants, disintegrating agents, solution retarders, wetting agents, adsorbents, lubricants and buffering agents.

14. The method of claim 11 wherein said pharmaceutically acceptable carrier further comprises selections from the group consisting of water, ethanol, polyols, oils, organic esters, lecithin, and suitable mixtures thereof.

15. A kit for treating disorders of the skin associated with free radical damage, said kit comprising an effective amount of the compound 2-(3-chlorophenylamino) phenylacetic acid or a pharmaceutically or cosmetically acceptable salt thereof and a dermatologically acceptable medium.

16. The kit of claim 15 wherein said dermatological acceptable medium comprises one or more cosmetic or manufacturing adjuncts selected from the group consisting of a sunscreen, a skin-lightening agent, a skin-tanning agent, a perfume, an opacifier, a preservative, a colorant and a buffer.

17. The kit of claim 15 wherein said dermatologically acceptable medium further comprises one or more selections from the group consisting of water, a buffered aqueous solution, a liquid emollient, or solid emollient, a silicone oil, an emulsifier, a solvent, a humectant, a thickener, a powder, or a propellant.
